(19) United States
(12) Patent Application Publication
(10) Pub. No.: US 2014/0316323 A1
(43) Pub. Date: Oct. 23, 2014

(54) DEVICE, USE THEREOF AND METHOD FOR REDUCING SCARRING OF WOUND

(71) Applicant: THE ROYAL INSTITUTION FOR THE ADVANCEMENT OF LEARNING / MCGILL UNIVERSITY, Montreal (CA)

(72) Inventors: Jonathan Kanevsky, Montreal (CA); Satya Prakash, Brossard (CA); Mirko Gilardino, Westmount (CA)

(73) Assignee: The Royal Institution for the Advancement of Learn ing/ Mcgill University, Montreal (CA)

(21) Appl. No.: 14/358,697

(22) PCT Filed: Nov. 16, 2012
(86) PCT No.: PCT/CA2012/050816

§ 371 Date: May 15, 2014

Related U.S. Application Data
(60) Provisional application No. 61/560,322, filed on Nov. 16, 2011.

(51) Int. Cl. A61F 13/00; A61F 13/02

(52) U.S. Cl. CPC A61F 13/00034; A61F 13/0243; A61F 2013/0028
USPC 602/53

(57) ABSTRACT

A scar reducing device for stretching skin having a closed wound therein is provided. The device is for reducing scar formation. The device comprises first and second fasteners removably attachable to skin regions located proximate to the wound. The device also comprises an extension mechanism cooperating with said first and second fasteners. The extension mechanism is movable between contracted and extended configurations. When the fasteners are affixed to the skin, the mechanism forces the first and second fasteners away from one another with a predetermined tensile force, stretching the skin proximate to the wound. Use of the device is also provided, along with a method for reducing scarring of a closed wound.

### FIELD OF THE INVENTION

The present invention relates to medical devices, uses and methods directed to the treatment of wounds, and more particularly concerns devices, uses and methods for improving wound scarring.

### BACKGROUND OF THE INVENTION

Cuts formed in a person's skin as a result of surgery, trauma, pathological conditions, burns, sports injuries and the like, typically heal in a manner which leaves scarring. While such scarring is often undesirable aesthetically, it can also result in other adverse effects, including loss of function, restriction of movement, reduced skin elasticity, psychological effects due to unsightly appearance and potentially a reduced quality of life.

As such, many attempts have been made to improve scar healing and reduce the adverse aesthetic effects of scarring.

The process of wound healing is known to be occurring in three sequential stages, which may overlap. The three stages are 1) the inflammatory stage, 2) the proliferative or granulation phase and 3) the remodelling phase.

During the inflammatory stage, inflammatory cells are sent to the injury site, and various cytokines, are released, preparing the wound site for the proliferation phase. This stage generally lasts from two to seven days, depending on the wound.

During the proliferative phase, fibroblasts arrive, proliferate and deposit collagen. At the same time, angiogenic factors are sent to the wound environment for stimulating the formation of new capillaries. Keratinocytes are also released across the wound. The proliferative phase is thus characterized by fibroblasts proliferation, as well as collagen production. The proliferative phase generally lasts from four days to several weeks, depending on the wound. Hypertrophic scars and keloids generally form during this phase.

During the remodeling phase, collagen forms and degrades and myofibroblasts contribute to increasing the tensile strength of the skin surrounding the wound. Granulation tissue deposition decreases as the cells responsible during this stage are suppressed; failure for this to occur often results in a hypertrophic scar. In the case of a hypertrophic scar, an overzealous healing response occurs, in which fibroblasts, small vessels, and collagen fibers are arranged in a nodular pattern. Alternatively, collagen can be inadequately replaced and, as a result, can form a pitted, unaesthetic appearance. The remodeling phase occurs at the end of the wound healing process, up to several weeks after the wound occurred.

Various pharmaceutical creams, powders, beads and other medicaments exist which, when applied to the wound site, are intended to reduce the effects of scarring by interfering with the proteins known to be involved in wound healing, skin growth and scar formation. The biological mechanisms involved in wound healing and scar formation are complex, and involve the interaction of many proteins in a series of steps. One particular protein (cytokine), TGF-β1, has been identified as playing a major role in scar formation. Particularly, reduced blood flow to the wound site causes hypoxia, which stimulates the deposition of extra-cellular matrix in response to the injury. The molecular signals that induce the localized production of such an extra-cellular matrix are regulated by TGF-β1. Therefore, the inhibition of TGF-β1 dependent pathways may prevent the profibrotic effects involved in scar formation.

Attempts have therefore been made to manipulate the mechanical regulators which regulate TGF-β1, such as tissue stretch or massage for example, as therapy for improved healing of large scars or diseases of excessive scarring, such as keloids for example. Several articles and studies discuss this matter, for example “Tissue Stretch Decreases Soluble TGF-b1 and Type-1 Procollagen in Mouse Subcutaneous Connective Tissue: Evidence from Ex Vivo and In Vivo Models” by Bouffard et al., from the University Of Vermont College Of Medicine. U.S. Pat. No. 6,756,518 B2, issued Jun. 29, 2004 to Gruskin et al., for example, discloses a method of reducing scar formation at a wound site by applying a TGF-β reducing amount of a cross-linked polysaccharide having a positive charge.

Many other more approaches have also been attempted to reduce localized scar formation at a wound site. For example, stitches, frames, adhesives, and other such devices which prevent relative motion of the skin around the area of the cut have been employed. Other devices are also known which draw the skin on opposing sides of a cut together, which maintain bandages in contact with a wounded area, or which attempt to immobilize skin the region of a cut in an attempt to reduce scarring as the cut heals. For example, U.S. Pat. No. 5,693,068, which issued Dec. 2, 1997 to Kuhlman, describes a scar-reducing frame structure which surrounds the cut and is removably attached to the skin such as to prevent motion relative to the healing cut, thereby allowing the cut to heal without being partially or fully reopened, which is said to damage the portions of the skin at the cut that had begun to heal. U.S. Pat. No. 8,183,428 issued May 22, 2012 to Gurtner, proposes a method of treating wound scarring by adhering an elastomeric material which compresses surrounding skin surface toward the wound, so as to reduce stress at the wound site.

Despite the attempts made to date to provide a device and/or method for facilitating healing of a skin wound such as to reduce scarring, there exists a continuing need for improvement in this respect.

### SUMMARY OF THE INVENTION

According to an embodiment of the invention, a scar reducing device for stretching skin having a closed wound is provided, so as to reduce scar formation. The device includes first and second fasteners removably attachable to skin regions located proximate to the wound, and an extension mechanism. The extension mechanism is movable between contracted and extended configurations. When the fasteners are affixed to the skin regions, the extension mechanism forces the first and second fasteners away from one another with a predetermined tensile force, thereby stretching the skin proximate to the wound.

According to an embodiment of the invention, there is provided a scar reducing device in which the extension mechanism includes first and second members aligned longitudinally with one another. Each of the members has inner and outer ends. The extension mechanism also includes a biasing element disposed between the inner ends of the members, the biasing element being able to generate a predetermined tensile force and allowing for the displacement of the first and second elongated members along a longitudinal axis, thereby moving the extension mechanism from a contracted configuration to an extended configuration. The first and second fasteners are removably attachable to skin regions located proximate to the wound.

Each of the fasteners includes a first connecting portion for attachment to the skin, and a second connecting portion affixed to the outer end of a corresponding member, the first and second connecting portions being manually attachable and detachable from one another.

When in use, the fasteners are affixed to the skin regions such that the longitudinal axis of the extension mechanism is substantially parallel to the wound, the biasing element forcing the first and second fasteners away from one another with the predetermined tensile force, thereby stretching the skin proximate to the wound.

According to another embodiment of the invention, use of the scar reducing device is made, for reducing scarring of a closed wound. Preferably, use is made for periodic and limited time intervals.

According to another embodiment of the invention, a method of reducing scarring of skin having a closed wound is provided, the wound having a segment with a substantially linear direction. The method comprises a step of periodically stretching the skin proximate the wound in a stretching direction which is substantially parallel the linear direction of the segment, with a predetermined tensile force.

According to another embodiment of the invention, the method comprising the steps of:
a) providing a scar reducing device as defined above;
b) fastening the first and second fasteners to skin proximate to the wound, the fasteners being aligned relative to the wound segment such that an axis passing through the fasteners is substantially parallel to the linear direction of the wound segment;
c) configuring the extension mechanism so as to generate a predetermined tensile force and disposing the extension mechanism for cooperation with the first and second fasteners, thereby stretching the skin surrounding the wound;
d) leaving the scar reducing device in place for a predetermined time interval;
e) removing the extension mechanism; and
f) repeating steps c) to e) periodically during the proliferative phase of wound healing.

According to another embodiment of the invention, the method comprises the steps of:
a) fastening the first connecting portions to the skin along or at opposite ends of the wound, said connecting portions being spaced apart by a given length interval;
b) placing the extension mechanism in the contracted configuration by displacing the elongated members toward one another, for compressing the biasing element and thereby generating the predetermined tensile force;
c) fastening the second connecting portions to said first connecting portions, respectively;
d) releasing the elongated members, thereby generating a static shear stretch through the wound in the skin;
e) leaving the extension mechanism in place for a predetermined time interval; and
f) detaching the first and second connecting portions of the fasteners, for removing the extension mechanism.

### BRIEF DESCRIPTION OF THE DRAWINGS

Other objects and advantages of the invention will become apparent upon reading the detailed description and upon referring to the drawings in which:

FIG. 1 is a top perspective view of a scar reducing device according to a preferred embodiment of the invention, the device being shown is fastened in position to skin having a closed wound therein.

FIG. 2A is a top view of the scar reducing device of FIG. 1, shown in an extended configuration, within its environment. FIG. 2B is a top view of the scar reducing device of FIG. 1, shown while being contracted. FIG. 2C is a top view of the scar reducing device of FIG. 1, shown in use. FIGS. 2D to 2F are side views showing different embodiments of fasteners.

FIG. 3 is a top view of a scar reducing device, according to another embodiment of the invention.

FIGS. 4A to 4C are side views of a scar reducing device, shown in different configurations and within its environment, according to yet another embodiment of the invention.

FIG. 5A is a top view of a scar reducing device, according to another preferred embodiment of the invention, shown within its environment. FIG. 5B is an enlarged side view of the device of FIG. 5A. FIG. 5C is a schematic top view of a scar reducing device, according to another preferred embodiment, shown within its environment.

FIG. 6 is a flow-chart representing a method of reducing scarring of skin having a closed wound therein, according with a preferred embodiments of the invention.

FIG. 7 is a flow-chart representing a method of reducing scarring of skin having a closed wound therein, according with another preferred embodiments of the invention.

FIG. 8 shows photographs of in vivo stretch models groups used in the Example described thereafter.

FIG. 9 is a graph showing measurements of extension force exerted by devices used in the Example described thereafter.

FIG. 10 is a graph showing morphological comparison of scars results using Vancouver Scar Scale obtained in the Example described thereafter.

FIG. 11 shows photographs of mice from five groups, used as per the Example described thereafter.

FIG. 12 are histological skin imaging pictures of studied mice used in the Example described thereafter.

FIG. 13 is a graph showing levels of TGF-β1 protein in cutaneous scars at day 20 for non-stretched and stretched tissue samples (N=30), used in the Example described thereafter.

### DETAILED DESCRIPTION OF PREFERRED EMBODIMENTS OF THE INVENTION

Scars formed in skin as a result of a healed wound, such as a wound formed due to surgery, trauma, pathological conditions or burns for example, are undesirable for a number of reasons, not the least of which are the adverse aesthetic effects that such scarring causes. The scar reducing device, according to an embodiment of the invention, is therefore provided to decrease the formation of scarring at such a wound site, thereby improving the aesthetic appearance of the wound, once fully healed. The term “wound” as used herein is intended to include any cut, rip, or other opening in the skin which may be caused by the aforementioned reasons.

The scar reducing device described therein differs significantly from previous attempts to achieve reduced scar tissue formation, and in fact functions in a manner fully contrary to what is currently proposed by existing devices currently available for wound scarring treatment. Current devices are designed such as to draw the skin on opposing sides of a wound together. Other devices work so as to immobilize the skin in the wound region.

As will be appreciated, the scar reducing device described below generates a shear, or stretching, force in the wound and/or surrounding skin which, when applied periodically during the proliferative phase of wound healing, that is, after the wound has closed but before scar formation has completed, reduces formation of scar tissue and therefore reduces the overall scarring left behind once the wound has fully healed. As such, the present device is intended for post-operative use on closed wounds to reduce scar formation.

The present devices works and is applied in a manner which is completely opposite to what other existing devices are promoting. An example of such an existing device is the Embrace™ Advanced Scar Therapy from Neodyne Biosciences™, which creates a stress shield around the wound, so as to avoid as much as possible any stress to be applied or transferred to the wound.

In contrast, the scar reducing device described therein allows for the application of tensile stress, periodically, that is, over several days, during limited time intervals, and with a predetermined tensile force, in the skin tissues of the wound and surrounding the wound. Indeed, it was found that applying stress to a closed wound, for periodic and predetermined time intervals, with a predetermined tensile force, leads to a reduction of the concentration of the TGF-β1 protein near the wound, which will in turn reduces collagen and fibroblast concentrations, resulting is a smaller scar of improved appearance.

In the following description, the same numerical references refer to similar elements. Furthermore, for the sake of simplicity and clarity, namely so as to not unduly burden the figures with several references numbers, not all figures contain references to all the components and features of the present invention and references to some components and features may be found in only one figure, and components and features of the present invention illustrated in other figures can be easily inferred therefrom. The embodiments, geometrical configurations, materials mentioned and/or dimensions shown in the figures are preferred, for exemplification purposes only.

### Scar Reducing Device

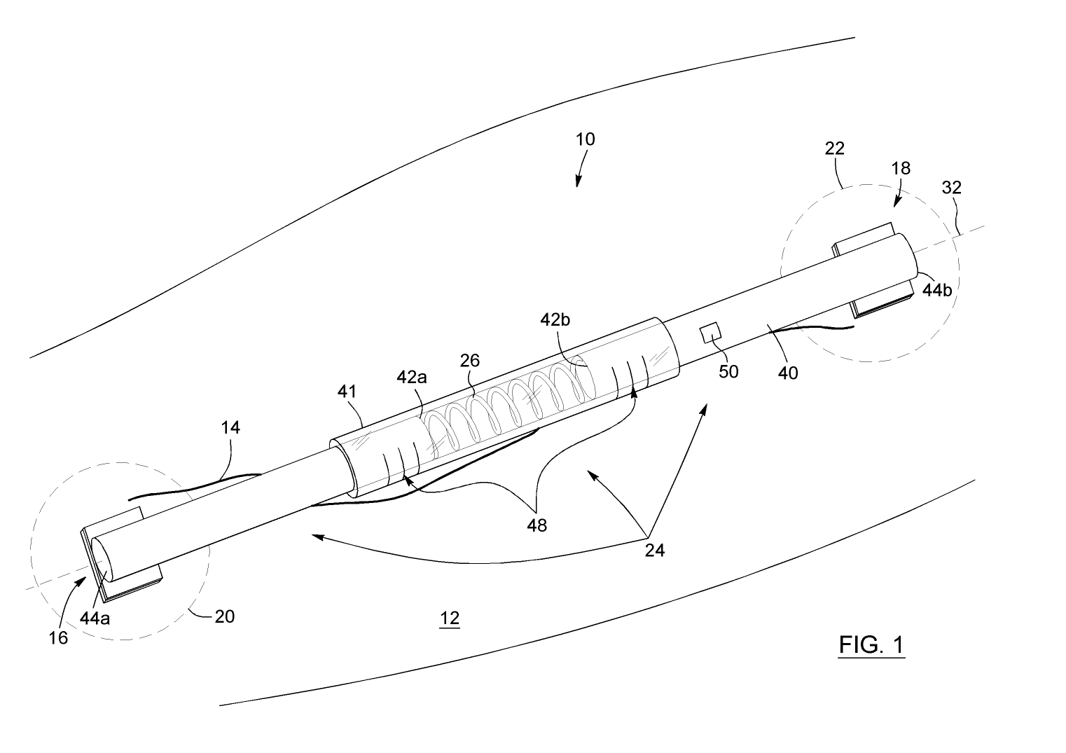

Referring to FIG. 1, a scar reducing device 10 for stretching skin 12 having a closed wound 14 is shown. The device 10 is used on skin 14 so as to reduce scar formation. The scar reducing device 10 includes first and second fasteners 16, 18 removably attachable to skin regions 20, 22 located proximate to the wound 14. The device 10 also includes an extension mechanism 24, which in this embodiment includes a biasing element 26 disposed between first and second members 38, 40.

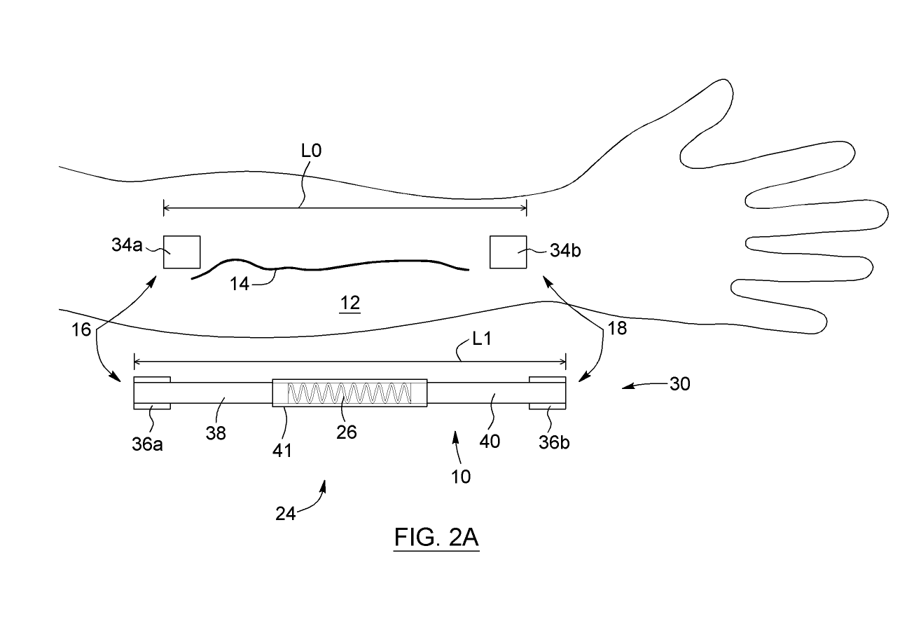

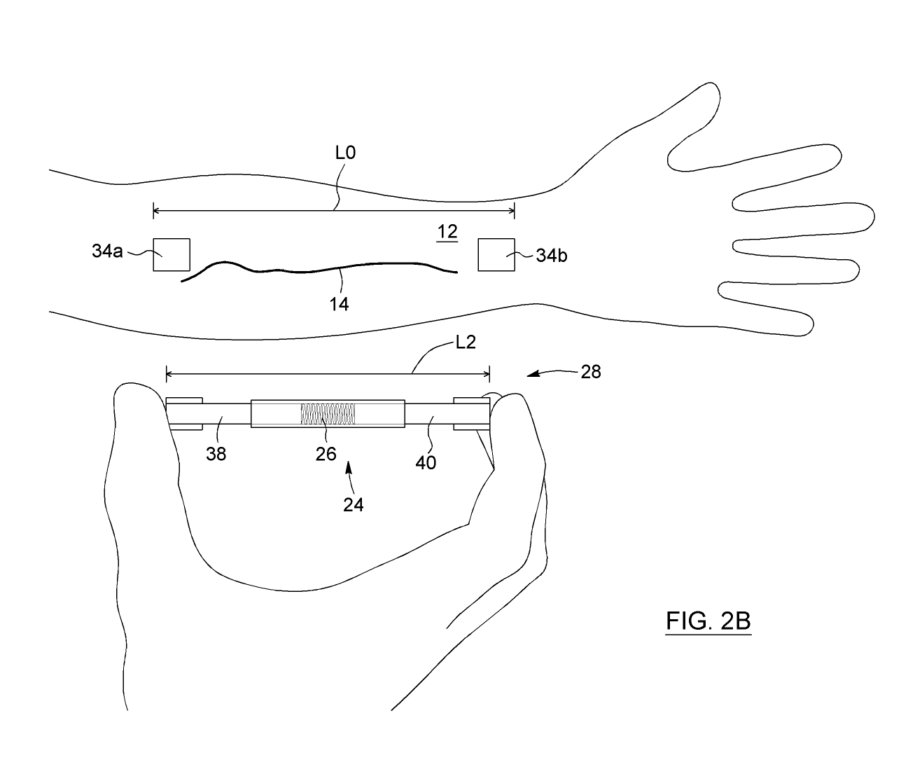

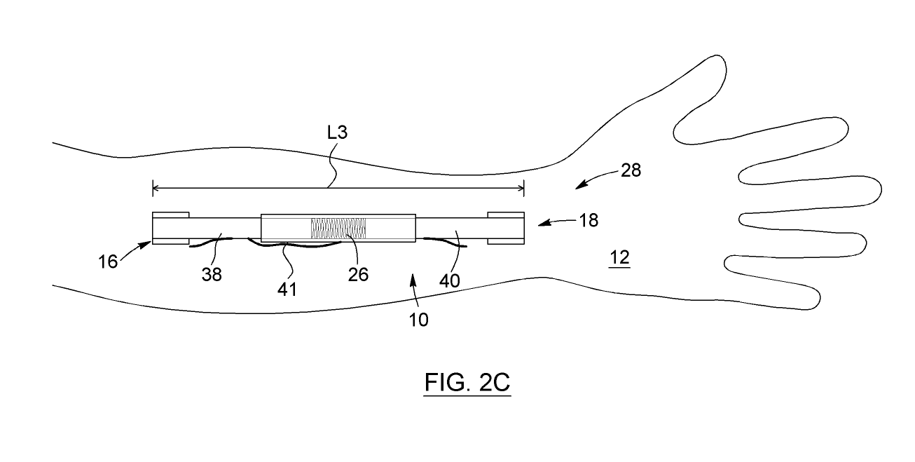

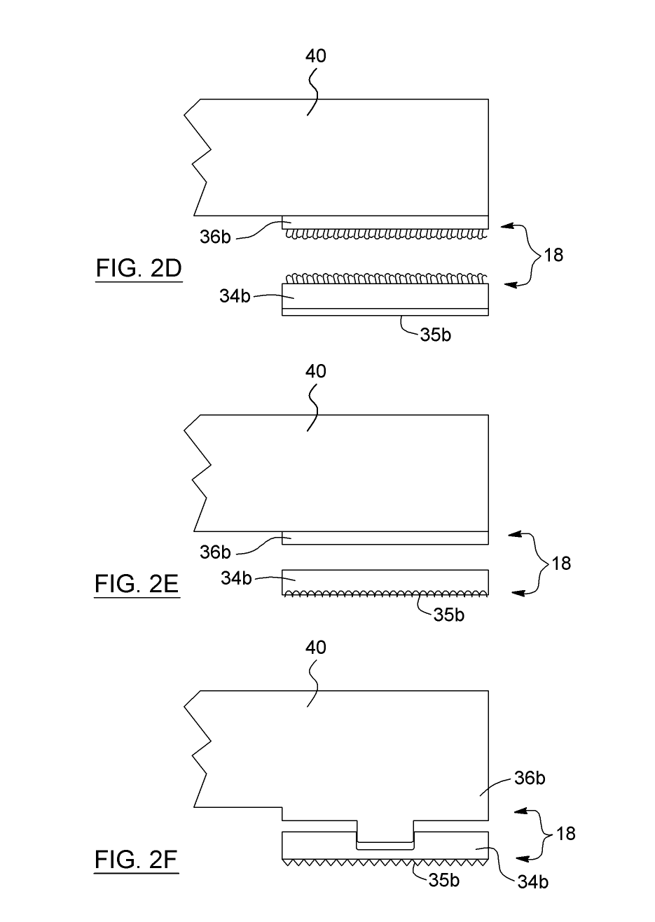

As best shown in FIGS. 2A and 2B, the extension mechanism 24 is movable between contracted and extended configurations 28, 30. In the contracted configuration 28, as per FIG. 2B, the extension mechanism stores energy for which will be used for stretching the skin 12 surrounding the wound 14, once attached to the skin. When in use, as shown in FIG. 2C, the extension mechanism 26 cooperates with the fasteners 16, 18 affixed to the respective skin regions 20, 22, forcing the first and second fasteners 16, 18 away from one another with a predetermined tensile force, thereby stretching the skin 12 proximate to the wound 14.

Referring to FIG. 2A, the extension mechanism 24 has a length L1 when in the extended configuration. As shown in FIG. 2B, the mechanism 24 can be contracted, or compressed, in the contracted configuration, reducing its length from L1 to L2. As per FIG. 2C, when in used and connected to the skin 12, the biasing element 26 urges the extension mechanism toward its extended configuration, the length of the extension mechanism increases up to L3. L3 is thus greater than L2, but smaller than L1. Consequently, a stretching of the skin surrounding the wound is obtained, stretching the skin from L0 (as per FIG. 2B) to L3 (as per FIG. 2C). As can be appreciated, the extension mechanism 24 acts to force the first and second members 38, 40 away from each other, such that the members 38, 40 normally extend outwardly, unless they are otherwise forced inwards.

Referring to FIG. 1, the first and second members 38, 40 are aligned longitudinally with one another. Each member 38, 40 has an inner end 42a, 42b and an outer end 44a, 44b. The biasing element 26 is disposed between the inner 42a, 42b of the two telescoping members 38, 40 within the surrounding tubular central body 41, to bias the telescoping members 38, 40 longitudinally apart. In this embodiment, the first and second members 38, 40 are elongated; however, other shapes can be considered for the members 38, 40. The fasteners 16, 18 are attachable to the outer ends 44a, 44b of the respective members 38, 40, but they could attach elsewhere on the members 38, 40.

Still Referring to FIG. 1, the extension mechanism 24 includes a tubular body 41 for housing the biasing element 26. The members 38, 40 are telescoping within the tubular body 41, whereby displacement of the elongated members 38, 40 toward one another allows compressing the biasing element 26. The first member 38 and the second member 40 engaged with each other such as to permit relative displacement between them along a longitudinal axis 32. This axis 32 is parallel to the stretching direction. The first and second members 38, 40 are matingly engaged for sliding axially within the tubular body 41. In other words, the first and second members 38, 40 are able to slide in a telescoping fashion in and out of the tubular body 41 in a direction parallel to the longitudinal axis 32.

In this particular embodiment, the two members 38, 40 are made of Teflon™. Of course, other material can be considered, such as metal or polymeric material. The tubular central body 41 preferably includes plastic tubing made from transparent material. Preferably, although not necessarily, the tubular body 41 is allowed to bend slightly (i.e. away from the surface of the skin) when the members 38, 40 are outwardly biased into their extended position.

Referring to FIG. 2A, the fasteners 16 includes a first connecting portion 34a, for fastening or attaching to the skin 12, and a second connecting portion 36a, affixed to the extension mechanism 24. Similarly, the fastener 18 includes a first connecting portion 34b, for attachment to the skin, and a second connecting portion 36b, affixed to the extension mechanism 24. Of course, in alternate embodiments, it can be considered to have the second connecting portions 36a, 36b integrally part of the outer ends of the extension mechanism 24.

Referring to FIGS. 2D to 2F, different embodiments of fasteners are shown. Only fastener 18 is illustrated, connected to the end of the member 40, since the fastener 16 has a similar construction. As can be appreciated, the pairs of first and second connecting portions 34a, 34b and 36a, 36b are manually attachable and detachable from one another.

The first connecting portion 34b is devised to be left on the skin 12 for several days during which the treatment will last, and preferably without removal during this period. The first connecting portions 34a, 36a can also be referred to as skin engaging fasteners. The first connecting portion 34b can be fastened to the skin using different types of connectors 35b. In FIG. 2D, an adhesive material is used as the connector 35b. In FIG. 2E, micro-sutures are provided while in FIG. 2F, micro-staples are provided. Hypoallergenic glue can also be considered.

Still referring to FIGS. 2D to 2F, the first and second connecting portions 34b, 36b are detachably connectable to one another thanks to a hook-and-loop attachment, such as Velcro™ or Dual-Lock™, an example of which is illustrated in FIG. 2D. Other types of detachable connections can be considered, such as magnets, as in FIG. 2E, and a male/female attachment, as in FIG. 2F. The fasteners can also consist of magnet button clip.

The adhesive force of the first connecting portions 34b must be able to resist a shear force relative to the skin that is greater than the predetermined tensile force generated by the biasing element. In other words, the connector which anchors the skin engaging fasteners to the skin must be stronger that the tensile force generated by the biasing member, otherwise the extension mechanism will over power the anchoring force of the skin engaging fasteners, causing detachment of the extension mechanism from becoming detached from the skin. The same reasoning applies to the detachable connection between the first portion 34b and the second portions 36b of the fastener 18. Once connected, the detachable first and second portions must be stronger than the tensile force applied by the extension mechanism.

Turning back to FIG. 1, the biasing element 26 is a spring able to generate the predetermined tensile force. Of course, other types of biasing element 26 can be considered, such as opposed-pole magnets, an elastomeric element, a resilient or curved metal or plastic plate, all of which resist axial compression. When these types of biasing elements 26 are used, the extension mechanism 24 is first placed in the contracted or compressed configuration, prior to being attached to the skin, substantially longitudinally along the wound 14. Once attached to the skin 12, the mechanism 24 is released so that the force accumulated in the biasing element is transferred to the skin, creating a shear force or stretch in the skin tissues surrounding the closed wound. The biasing element 26 is selected such that its elastic constant is able to exert the predetermined level of tensile force when compressed over a given length.

The biasing element 26 may be detachably fixed to the inner ends 42a, 42b of the first and second members 38, 40 such that the biasing element 26 is replaceable. This may be desirable, for example, if the biasing element 26 is to be removed and replaced with another having a different spring constant, that is stiffer or softer, as required. This allows selection of the biasing element 26 so the extension mechanism 24 may generate adequate predetermined tensile force. This may also be advantageous if the biasing element 26 is to be maintained in the device 10, but the two members 38, 40 are to be replaced, as may be desirable if the device is to be used several times, with one or more different patients.

In the alternate embodiment wherein the biasing element 26 is magnetically driven, permanent magnets for a selected magnetic force and having opposed poles are fixed to each of the inner ends 42a, 42b of the members 38, 40, such that they repel each other thereby forcing the members 38, 40 longitudinally outwardly within the tubular body 41. Alternately, electro-magnets can be used which are controlled such as to repel each other for a predetermined amount as required to create a selected overall length of the device.

Yet other types of resilient elements can be considered, such as an endless screw or a rack-and-pinion mechanism. In such cases, it is required to first attach the extension mechanism 24 to the skin engaging fasteners first, while the mechanism is in the contracted configuration, and then once attached, to move the mechanism 24 from a contracted to an extended configuration, so as to stretch the skin tissues, for example with a screw, so as to control the level of force, or stretch, applied to the skin.

Preferably, the biasing element 26 generates equal and opposed longitudinally acting force on the members 38, 40 which have the effect of forcing an expansion of the scar reducing device 10.

The biasing element 26 thereby, when the opposed outer ends 44a, 44b of the members 38, 40 are fastened to the skin 12 as described further below, generates a substantially constant shear force in the skin 12 surrounding the closed wound 14, and thus in the wound 14 itself. When such a static shear force is imposed on the skin 12 it stretches, reducing the concentration of TGF-B1 thus suppressing the activity of collagen-producing cells occurs in the healing wound. As such, scar formation can be decreased. This stretch or shear force applied to the wound 14 by the present scar reducing device 10 is preferably applied during the proliferative phase of wound healing, which begins after the wound has closed but before the scar has completed formation the proliferation phase, which follows immediately the inflammation phase, may begin from 5 to 21 days after the initial wound formation, and can vary from one patient to another and from one wound to another. It is of note that the terms “shear force” and “shear stretch” are used herein interchangeably, and are intended to comprise any stretching of the skin produced by a force applied in a plane substantially parallel to the skin surface.

The biasing element 26 may be free to axially move relative to the surrounding central body 41, whereby the entire sub-assembly formed by the two members 38, 40 and the linking biasing element 26 disposed there in-between is longitudinally displaceable within the tubular central body 41. Alternately, however, the biasing element 26 may be located in position at a central point within the tubular central body 41.

Additionally, although the first and second members 38, 40 and the tubular body 41 are depicted in the figures as having a circular cross-sectional shape, it is to be understood that in alternate possible embodiments, one or all of these components may have any one of a rectangular, circular, square, trapezoid, toroid, oval cross-sectional shape. The first and second members 38, 40 may also be composed of several, linked-together, portions rather than being formed from a single integral rod as depicted.

Preferably, the biasing element is able to generate a predetermined tensile force between 100 and 2000 g. Still preferably, this interval is between 250 g and 1200 g, and more preferably, between 400 and 800 g. The device 10 may also preferably includes a controlling mechanism 146, allowing to controllably varying the tensile force of the biasing element.

Still preferably, the device 10 includes a sensor 50 for measuring one or several biometric characteristic(s) of the skin in the region of the wound, such as temperature, humidity, and tensile strength of the skin. The sensor 50 can also measure static shear stretch generated by the extension mechanism in the skin tissues. Still preferably, the sensor can measure a concentration of collagen at the skin surface or within the skin. The sensor may also include the indicator in communication therewith, which is operable to indicate at least a level, if not an exact value, of the measured characteristic, such as a general level of the static shear stretch applied to the wound for example.

In a preferred embodiment, the sensor 50 also includes a wireless signal generator operable to send readings from the sensor to a remote receiver (connected to a server and/or computer, for example) for data collection and storage. This permits either the patient or physician the ability to monitor the progress as the wound heals, in order to be able to better track the formation of scar tissue. Additionally, the force produced by the device 10 can be adjusted by the patient or physician based on the information received from the sensor 50. Usage of the device 10 can thus be modified as required, for example used for longer periods or at a higher frequency, if deemed necessary in order to reduce the scar formation to a desirable level. As skin characteristics tend to vary between individuals (ex: different skin thicknesses, tensile strengths, etc.), the ability to monitor wound healing and adjust the amount of shear force applied to the wound/skin by the present device is advantageous. With such preferred embodiment, the device 10 is not only able to deliver stretch to the wound site, without requiring the patient to apply any external force to the device during use, but also to adjust it according to the patient's specific skin profile.

Alternatively, or in combination with the controlling mechanism, the device 10 includes an indicator 48, indicative of the tensile force of the biasing element. As shown in FIG. 1, the indicator 48 can consists for example, in graduation marks on the extension mechanism, indicating the level of compression of the biasing element, the predetermined force of the biasing element being function of this level of compression. A digital display can also be considered to indicate the predetermined tensile force. This indicator may also comprise graduation markings on the tubular body 41 or the telescoping members 38, 40 calibrated such that their relative position is known to produce a given shear stretch value, for example.

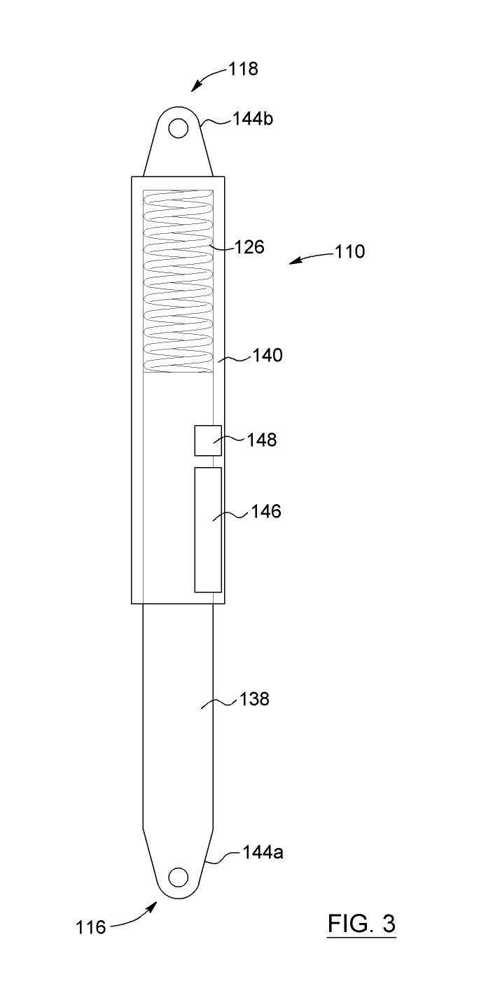

Referring now to FIG. 3, a scar reducing device 110 in accordance with an alternate embodiment operates substantially as per the scar reducing device 10 described above. The scar reducing device 110 comprises an elongated body which, however, includes only a first telescoping member 138 and a second telescoping member 140 which are matingly received within each other, rather than being matingly received within a tubular central body as per the device of FIG. 1. Accordingly, the first telescoping member 138 is a rod which mates within the larger-diameter tubular second telescoping member 140. Relative longitudinal displacement of the first and second telescoping members 138, 140 is permitted, such as to displace the device 110 from a fully extended configuration to an at least partially contracted, or compressed, configuration. The device 110 also includes an extension mechanism which resists this compression of the device from the extended to the contracted configuration, having a biasing element 126 disposed between an inner end 142a of the first telescoping member 138 and a close base end 144b within the tubular cavity of the second telescoping member 140. The biasing element 126 therefore acts to force the first and second telescoping members 138,140 longitudinally apart, and thus to generate a static shear stretch in the skin across the wound when the device 110 is fastened in place on the skin over the wound. Each of the outermost ends 144a, 144b of the first and second telescoping members 138,140 have fasteners, such as anchor points 116, 118 therein, which are adapted to be attached in a releasable manner to skin engaging fasteners (not shown in FIG. 3) which would include hook for attachment with the anchor points.

The scar reducing device 110 is used in the same manner as the device 10 described above. The device 110 is compressed in the contracted configuration until the desired predetermined tensile force is accumulated in the biasing element 126. A controlling mechanism 146 allows controlling the force accumulating in the biasing element. For example, the controlling mechanism can comprise a serrated groove extending along the member 140, and a releasably engaging pin cooperating with the serrated groove, for allowing control of the level of compression of the extension mechanism. An indicator 148 can also be placed on the outer surface one of the members 138, 140, so as to provide an indication of the compression of the biasing element. In use, the fasteners are fastened to the skin on opposite sides of the wound during a proliferative phase of the healing thereof, such that the static shear stretch generated in the wound and/or skin reduces scar tissue formation in the wound.

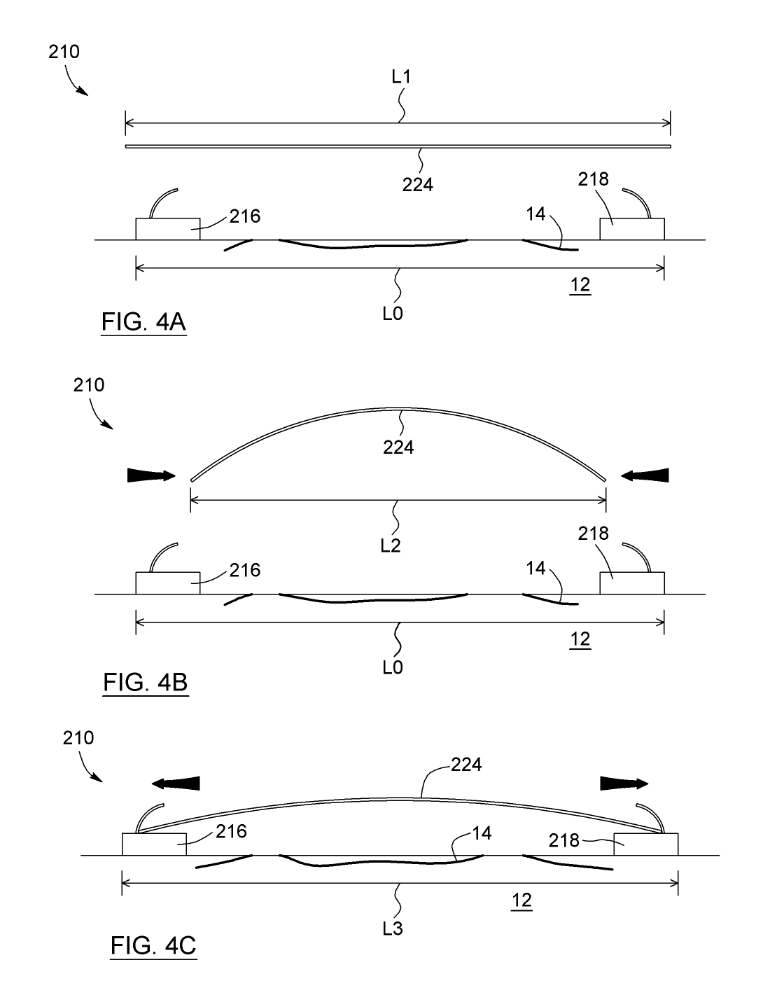

Referring now to FIG. 4A to 4C, a scar reducing device 210 in accordance with yet another alternate embodiment is illustrated. The scar reducing device 210 comprises first and second fasteners 216, 218 attachable in a removable manner to the skin, and an extension mechanism 224, for cooperating with the fasteners 216, 218. In this case, the extension mechanism is a flexible element, such as a rod or a plate, which is movable, or configurable, between contracted and extended configurations. The flexible element 224 can be made of metal, alloy, plastic or composite material selected with specific flexibility characteristics.

In FIG. 4A, the extension mechanism is shown in its extended configuration. When moved from the extended to the contracted configuration, as in FIG. 4B, the plate accumulates elastic potential energy, which can be transferred back to the fasteners 216, 218, so as to stretch the skin 12. While keeping the biasing element 224 in this contracted configuration, it is placed in between the fasteners 216, 218, which are spaced apart by a given length L2, as shown in FIG. 4C. The biasing element 126 forces the fasteners 216, 218 away from one another with the predetermined tensile force, thereby stretching the skin proximate to the wound. The rod thus has a first length L1 when in the extended configuration, and it can be curved to a second length L2 in a contracted configuration, L2 being smaller than L1. The rod is curved, or squeezed, for generating the predetermined tensile force. Once attached to the fasteners 216, 218, the rod extends to L3, which is smaller than L1, but greater than L2. Of course, The fasteners 216, 218 respectively include skin connecting portions for fastening to the skin 12, and receiving portions, for receiving ends of the rod when curved in the contracted configuration.

The length L0 by which the fasteners 216 and 218 are spaced apart, the length L1 of the flexible rod 224 and its resiliency are selected so that the force exerted by the rod 224 when disposed between the fasteners 216, 218 corresponds to a predetermined tensile force. In other words, the resiliency of the biasing element 224, which is characterized by an elasticity constant or elastic coefficient, and its length L1, is selected so than when compressed to L3, it exerts the predetermined tensile force. Of course, the predetermined tensile force can be a range, or interval, of force.

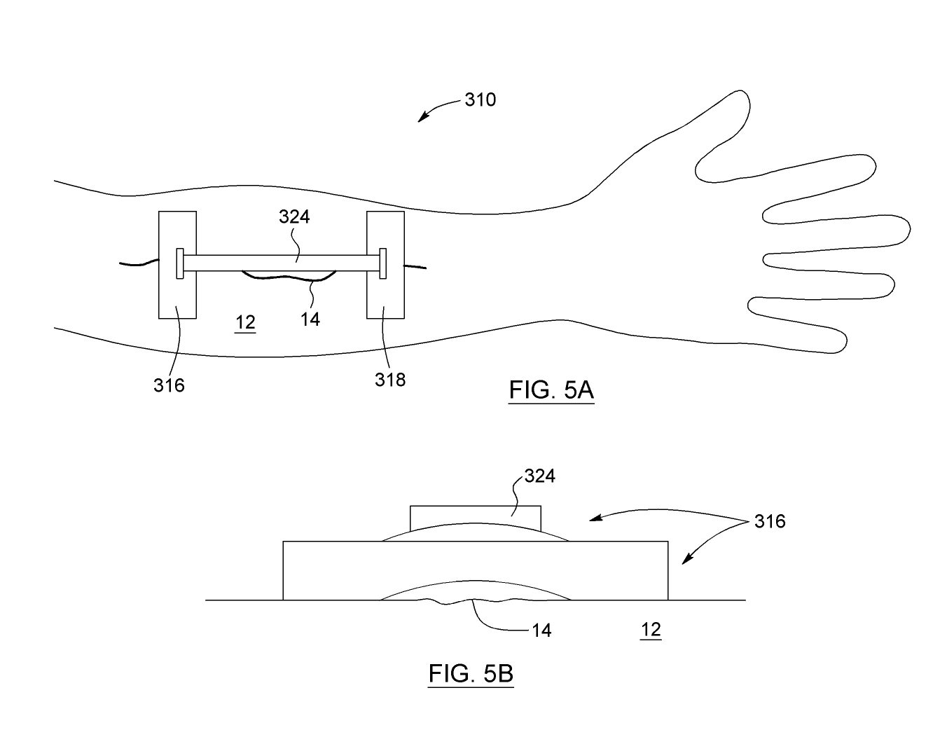

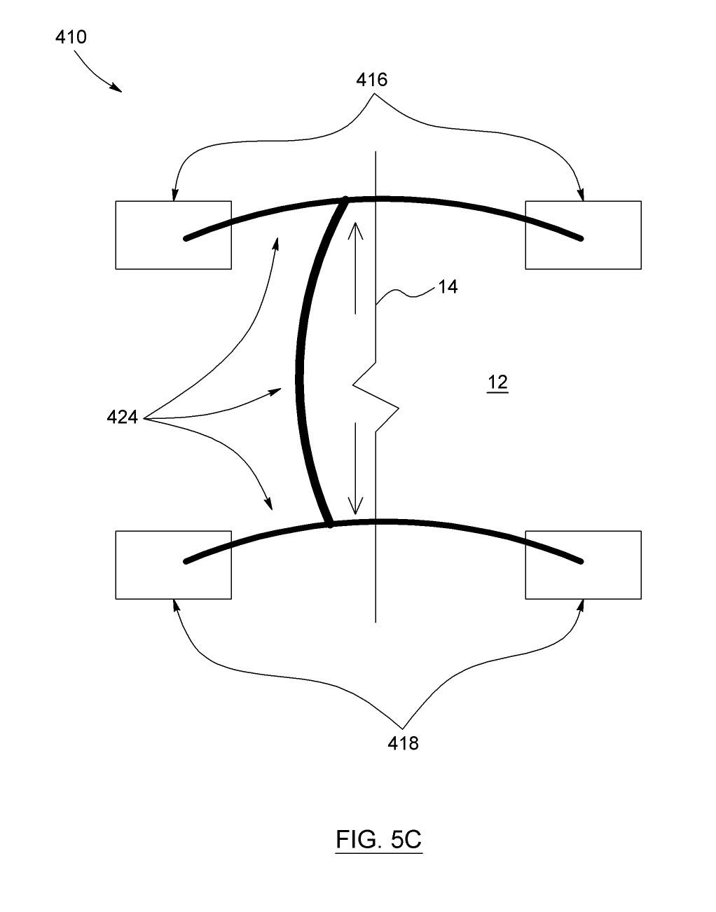

Referring now to FIGS. 5A and 5B, yet another embodiment of the scar reducing device 310 is shown. In this case also, the extension mechanism 324 is a flexible rod. The fasteners 316, 318 have its bottom side (ie the skin engaging side) curved, such as to span over the wound 14, without touching it. This construction of the device can be advantageous in cases where the wound is long, and several devices must be placed along the wound.

Referring to FIG. 5C, yet another embodiment of the device is shown. In this case, the extension mechanism 424 of the device 410 is a tension rod having three segments, which can be integrally formed or not. The fasteners 416, 418 each include two connecting portions, for location of opposed sides of the wound 14. When attached to the fasteners 416 418, the extension mechanism 424 is urged to move from a contracted to an extended configuration, forcing the fasteners 416, 418 away from one another with a predetermined tensile force.

All of the embodiments described above may be fully disposable, such that it is intended to be used on a single-use basis by a patient who installs the device himself or herself during the healing process of a skin wound. Accordingly, the materials chosen for the device may be selected in consequence.

Of course, any of the embodiments described above can form a kit, which includes the different components such as the extension mechanism and/or fasteners. Preferably, a graduated ruler can be included in the kit for facilitating positioning of the fasteners on the skin, and ensuring that they are spaced apart by a predetermined length L0.

### Use of the Medical Device and Method for Reducing Scarring of a Closed Wound

The scar reducing device as described herein may therefore be used in the following manner, in accordance with the described method for facilitating the healing of a skin wound such as to reduce scarring.

### Use of the Scar Reducing Device and Method of Reducing Scarring

The method is devised to be performed on a closed wound, which has at least one a segment with a substantially linear direction. By closed wound, it is meant a wound which as at least one segment closed, the segment being able to resist tensile forces of less than 250 g without opening. By substantially linear, it is meant a generally linear orientation. The wound can comprise only one segment, which is substantially linear or it can comprises several segments, where if the general aspect of the wound is not linear, at least one or some segments have a general linear profile.

According to one aspect, the method includes a step of periodically stretching the skin proximate the wound. By periodically, it is meant that the stretching is repeated several times during a given period. The given period preferably begins toward the beginning of the proliferative phase of the wound scarring and ends prior the end of the remodeling period. Still preferably, the treatment begins towards the end of the proliferative phase of the wound, and ends toward the beginner of the remodelling phase.

The treatment period can begin about 5 days after the date of injury date and last until about 45 days after the date of injury. Alternatively, the period begins from 10 to 15 days after the date of injury, and end from 20 to 40 days after the date of injury. Of course, the period can vary according to the severity of the wound, the skin type of the patient, the length of the wound, and the likes.

The stretching is made in a stretching direction which is substantially parallel to the linear direction of the wound or wound segment. For example, the angle formed between the stretching direction and the wound segment direction can vary between 0 and 45 degrees.

The stretching of the skin is made with a predetermined tensile force. The tensile force can vary between 100 g to 2000 g, and is preferably between 250 g and 1200 g, and still preferably between 400 and 800 g. As it will be explained in the Example provided thereafter, a tensile force which is too small or too great will not lead to reducing of the scar. In cases where the tensile force applied is too great, that is, outside the predetermined ranges described above, scarring of the wound is likely to be increased, rather the decreased, as desired.

During the treatment period, the stretching is performed for predetermined time intervals. A time interval is relatively short, and preferably varies from about 5 to about 20 minutes. Still preferably the time interval is between 8 and 15 minutes. Preferably, the stretching step is made twice a day. The stretching step is preferably performed about every 12 hours. It can also be considered to perform the stretching step only once a day, and up to four times a day.

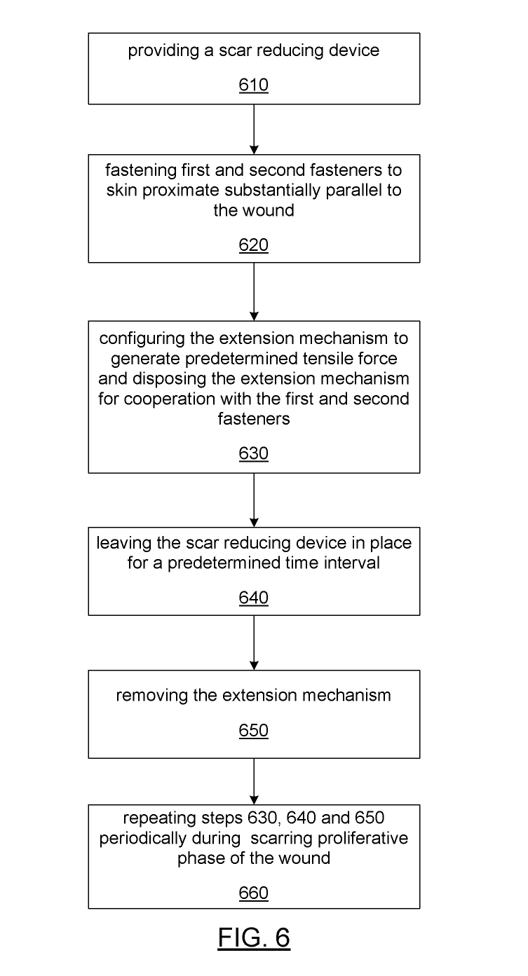

Referring now to FIG. 6 and also to FIG. 4A to 4C, a preferred embodiment of the method of reduced scarring of skin having a closed wound will be described. In a first step 610, scar reducing device 210 is provided. Then, as a second step 620, the fasteners 216, 218 are then fastened to the skin, proximate to the wound. The fasteners 216, 218 are aligned such that an axis passing through them is substantially parallel to the direction of the wound or wound segment, and preferably at angle varying between 0 to no more than 45 degrees direction of the wound. They are also spaced apart by a predetermined distance L0. In step 630, the extension mechanism 224 is configured so as to generate a predetermined tensile force, for stretching the skin surrounding the wound. In this particular case, configuring the extension mechanism 224 means compressing the flexible element 226 sufficiently enough to be able to insert it between fasteners 216, 218. Of course, it is possible to first insert one end of the flexible element 224 into the fastener 216, then press or squeeze the element 226, and insert the second end of the flexible element 224 into the fastener 218. In step 640, the device 210 is then left in place for a predetermined time interval; preferably from 5 to 20 minutes. In the next step 650, the extension mechanism is removed. The steps 630, 640 and 650 are repeated periodically during the proliferative phase of the wound, or preferably during a period which extends from 7 days up to 40 days after the date of injury or incision.

Or course, in an embodiment of the method in which an endless screw, or a rack and pinion system is used as the scar reducing device, the step 630 of configuring the extension mechanism consists of first attaching the extension mechanism to first and second fasteners, and then extending the mechanism to its extended configuration, once affixed to the skin.

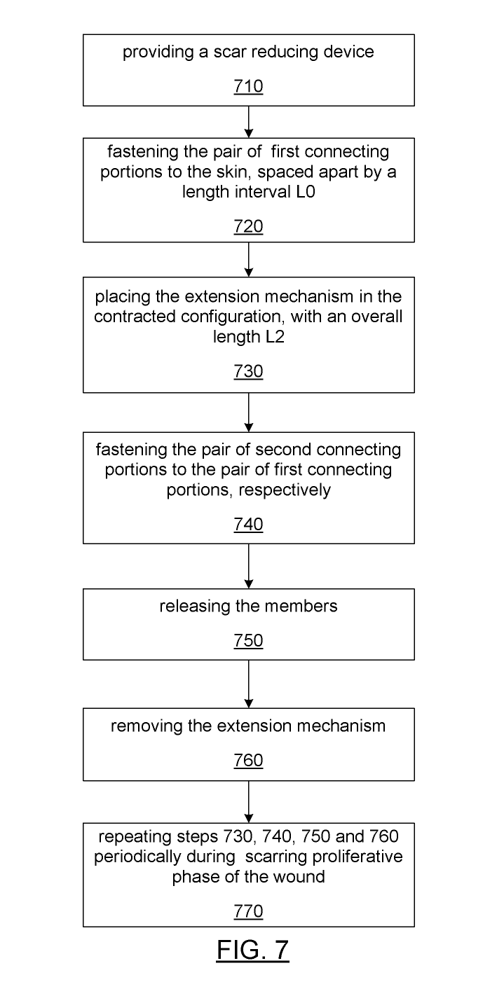

Referring now to FIG. 7 and also to FIGS. 2A to 2C, another embodiment of the method will be described. In step 710, a device such as device of FIGS. 2A-2C is provided, with telescoping members. Then, in step 720, the first connecting portions 34a, 34b are fastened to the skin along or at opposite ends of the wound, the connecting portions being spaced apart by a given length interval L0. In step 730, the device 10 is placed, which in this case means it is compressed, in its contracted configuration, such as shown in FIG. 2B. The elongated members 38,40 are displaced toward one another, thereby generating a predetermined tensile force. While in the contracted configuration, as per step 740, the extension mechanism 24 is fastened by connecting the second connecting portions 36a, 36b to the first connecting portions 34a, 34b. In step 750, the members 38, 40 are released, thereby generating a static shear stretch through the wound in the skin. The elastic energy accumulating within the spring 26 is transferred as tensile force to the fasteners 16, 18 which in turn is transferred as static shear to the skin tissues surrounding the wound. In step 760, the extension mechanism is left in place for a predetermined time interval, which is preferably between about 5 to about 20 minutes. When the time interval has lapsed, in step 770, the extension mechanism is removed by detaching the first and second connecting portions from one another. Preferably, the method is performed during the proliferative period of the wound-healing. Preferably, steps 730 to 770 are performed twice a day. As it can be appreciated, by then releasing the outer ends of the two telescoping members 38, 40, to create a tensile force between the telescoping members, a static shear stretch through the wound in the skin is thereby generated, which reduces scar formation as the wound heals during the proliferative phase of wound healing, i.e. after the wound as initially closed, but before scar formation has completed.

It is to be understood that the force generated by the biasing element of the scar reducing device is selected to be insufficient to open a healing wound, even if the device is applied to the wound as described above too soon, that is before the proliferative phase of wound healing (roughly 21 days after the initial wound creation).

Although the specific duration and frequency of use of the scar reducing device can be varied and determined as required by the directing physician, in at least one possible usage contemplated, the device is preferably applied to the wound for periods of only 15 minutes, two times per day.

The device described herein may be disposable, and thus may only need to be used once or at least a limited number of times before being discarded. For purposes of reducing possible infection of the wound, such a one-time use of the device may also be desired. However, the device 10 may also be configured for multiple re-uses. As such, in at least one embodiment, all parts of the scar reducing device may be composed as to allow for sterilization such as gamma irradiable or vapour sterilization, heat sterilization.

### Example

Studies have shown that tension at the site of the wound can worsen scar formation, such as described by Huang C, Akaishi S, Ogawa R. in the article entitled “Mechanosignaling pathways in cutaneous scarring” (Archives of dermatological research. 2012. Epub 2012 Aug. 14). A demonstration that stress can induce or promote hypertrophic scar formation is also made in U.S. Pat. No. 8,183,428 to Gurtner. However, recent studies suggest that mechanical forces acting on a scar can be a factor in scar formation and may decrease scar formation. Bouffard et al. describe a series of investigations in article entitled “Tissue stretch decreases soluble TGF-beta1 and type-1 procollagen in mouse subcutaneous connective tissue: evidence from ex vivo and in vivo models. Journal of cellular physiology. 2008; 214(2):389-95. Epub 2007 Jul. 27.” Massage and mechanical manipulation have also been often recommended to patients for treatment of scars.

The study presented below was conducted based on the hypothesis that tissue stretch parallel to a scar may decrease scar formation. To investigate this we set out to create a device and animal model in which to investigate the effects of longitudinal tissue stretch on scar formation.

### Research Methods

### Device Development and Standardization

A scar stretch device was designed that can easily attach and detach from skin. The components of the device included a skin adhesive mechanism and an extension force mechanism. The device prototypes were constructed using inert materials: Steel spring, polyvinyl tubing, Teflon rods, and an adhesive. Three different spring strengths for the scar stretch devices were created and labeled as 0.5×, 1× and 2× to investigate a dose response. The devices were standardized to ensure similar extension force using a small force gauge from Jonard Industries.

### In Vivo Scar Stretch Study Design

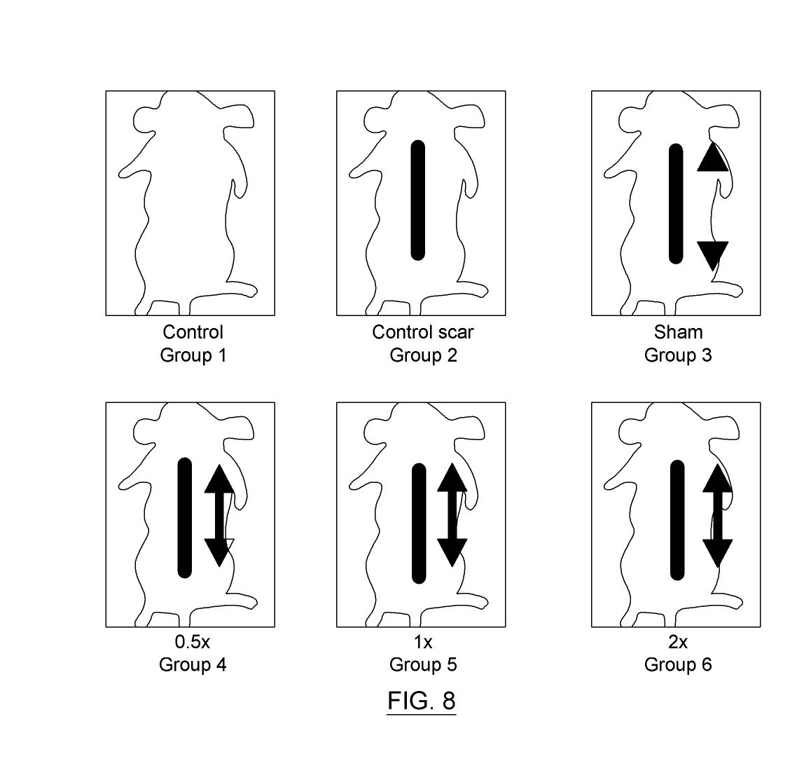

The experimental protocols used in these experiments were approved the McGill University Ethics and review board. All mice were female Balb/C weighing 19-21 g. Thirty mice were divided equally into 6 groups, as per FIG. 8.

Group 1 included control mice without scar. Group 2 mice received a dorsal incision and no treatment. Group 3 mice received a dorsal incision and treatment with a sham device. Group 4 mice received a dorsal incision and treatment with a stretch device that was half strength (0.5× group). Group 5 mice received a dorsal incision and treatment with a stretch device that was full strength (1× group). Group 6 mice received a dorsal incision and treatment with a stretch device that was double strength (2× group).

It is also worth mentioning that correlations can be established between mice data and human data, for example as described in “A Review of the Role of Mechanical Forces in Cutaneous Wound Healing” from Riaz Agha et al. (Journal of Surgical Research 171, 700-708 (2011)).

### Surgical Procedure

Under isoflurane anesthesia, 24 mice were shaved and received a three centimeter incision in the middle of the back at the level of the scapula. A microsurgery blade was then used to cut the subcutaneous tissue attachments between the pannicular muscle and the back muscles (0.5 cm of undermining lateral to the incision bilaterally). Incisions were closed primarily with Steri-strips™. One mouse in the control scar group (group 2) was eliminated due to wound dehiscence.

### In Vivo Tissue Stretch Method

On day five post-incision mice underwent stretching of the trunk for 10 minutes, twice a day, for 10 days. All mice underwent anesthesia with isoflurane and mice in groups 3 to 6 underwent stretch treatment with device. The device was aligned in parallel over the scar and attached only during the 10 minute stretch period. After 5 days after the last stretch treatment, ie (20 days post-incision, all mice were euthanized. The skin of the back was excised and fixed for 2 h in 3% paraformaldehyde in phosphate buffered saline (PBS). Skin samples were also frozen and stored for biochemical analysis.

### Morphologic Scar Assessment

Photos of scars 15 days after beginning tissue stretch, ie 20 days post incision, were qualitatively analyzed using the Vancouver Scar Scale.

### Histologic Analysis

Following fixation, cutaneous tissue samples of 1 cm×1 cm and 30-50 μm thickness, centered 1 cm lateral to the spine at the level of the surgical incision, were taken and mounted on glass slides. Slides were stained with Masson-Trichrome to show collagen and described using light microscopy.

### Cutaneous TGF-B1 Assay

Thawed skin samples were homogenized and immediately assayed for (1) TGF-β1 protein using a human TGF-β1 ELISA assay (R&D Systems, Minneapolis, Minn.) including sample acidification with 1N hydrochloric acid for activation of latent TGF-β1.

### Statistical Methods

Analyses of variance (ANOVA) were performed to test for differences of TGF-β1 level between treatment groups. ANOVA was used to analyze the effects of stretch on TGF-β1 concentrations after five days after 10 consecutive days of stretch therapy. For these analyses, TGF-β1 data were log transformed prior to analysis in order to satisfy the normality and homogeneity of variance assumptions associated with the ANOVA. Statistical analyses were performed using SAS statistical software (PROC MIXED). P values <0.05 were considered statistically significant.

### Results

### Device Development and Standardization

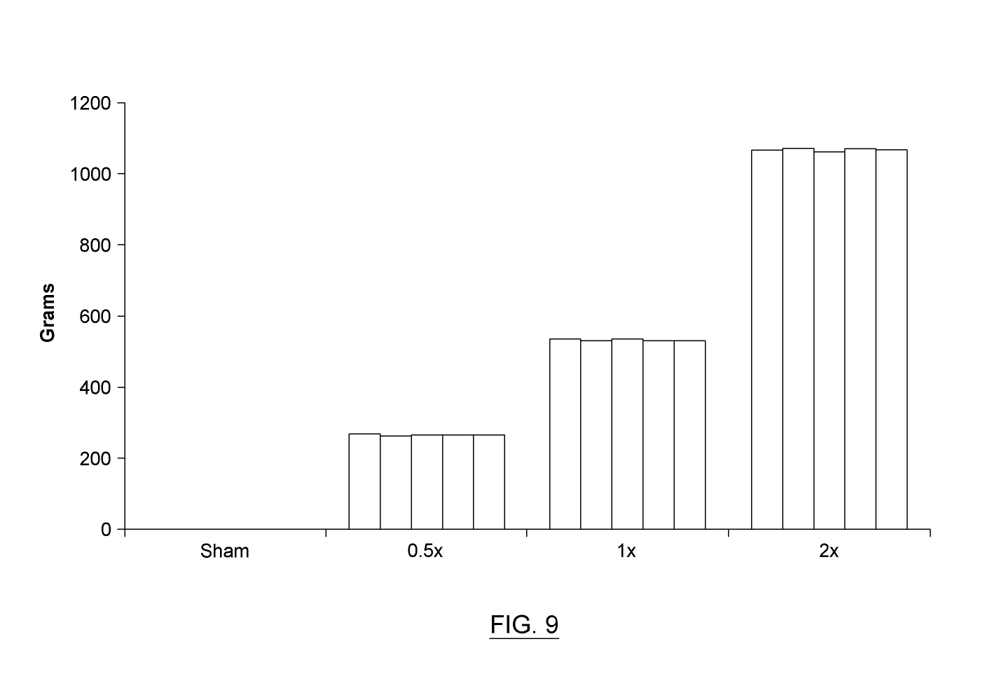

A total of 20 devices were created and grouped into four different stretch strengths categories. The force produced of by each device, except the sham was measured. FIG. 9 is a graph of the measured extension forces, in grams, exerted by each device using a force gauge. The strength categories were (1) A sham device, which consisted of the device without any spring extension force mechanism that produced no extension force (2) A 0.5× device which exerted a mean force of 265.6 g±1.5 g (3) A 1× device which exerted a mean force of 532.4±1.8 g (4) A 2× device which exerted a mean force of 1068.4 g±3.4 g.

### Morphologic Scar Assessment

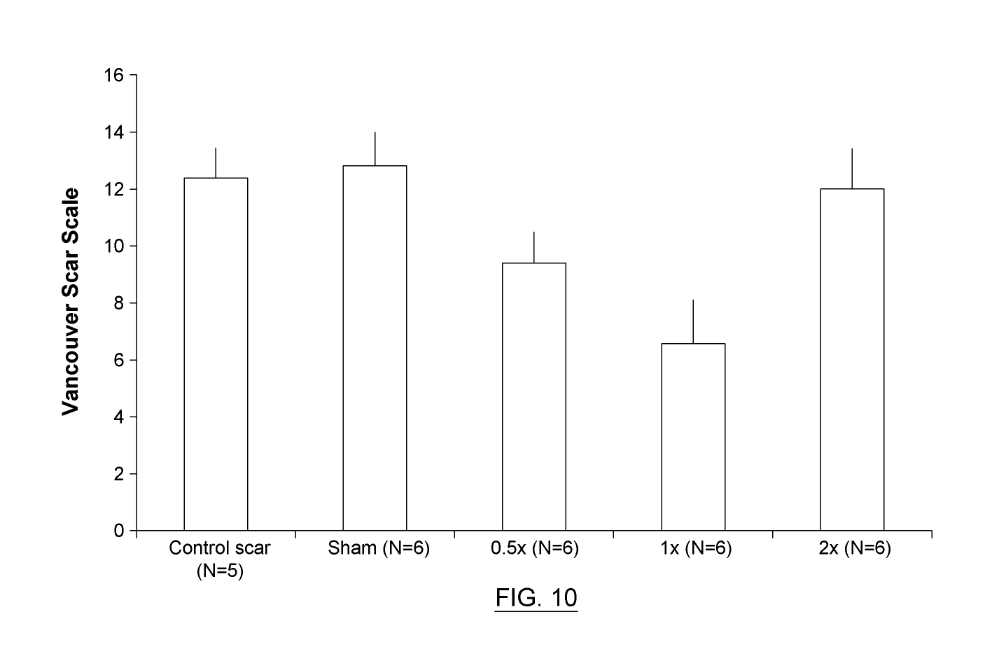

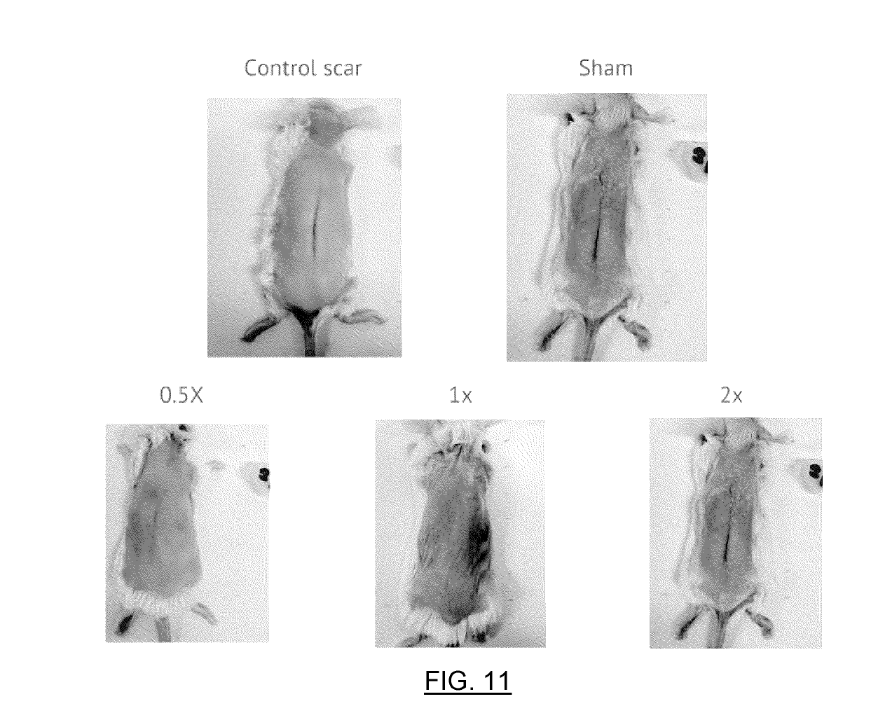

Photos of scars 15 days after beginning tissue stretch, ie 20 days post incision were qualitatively analyzed using the Vancouver Scar Scale, as shown in FIG. 10, which shows a morphological comparison of scars using Vancouver Scar Scale). Control scars averaged 12.4±1.0, Sham scars 12.8±1.16, 0.5× stretch treatment group 9.4±1.0, 1× stretch treatment group 6.6±1.5, 2× stretch treatment group 12±1.4 (p<0.05) On examination 20 days post incision (5 days after last stretch treatment) scars remained most visible in the sham, control scar, and 2× treatment groups, as can be seen in photographs of FIG. 11.

### Qualitative Histologic Analysis

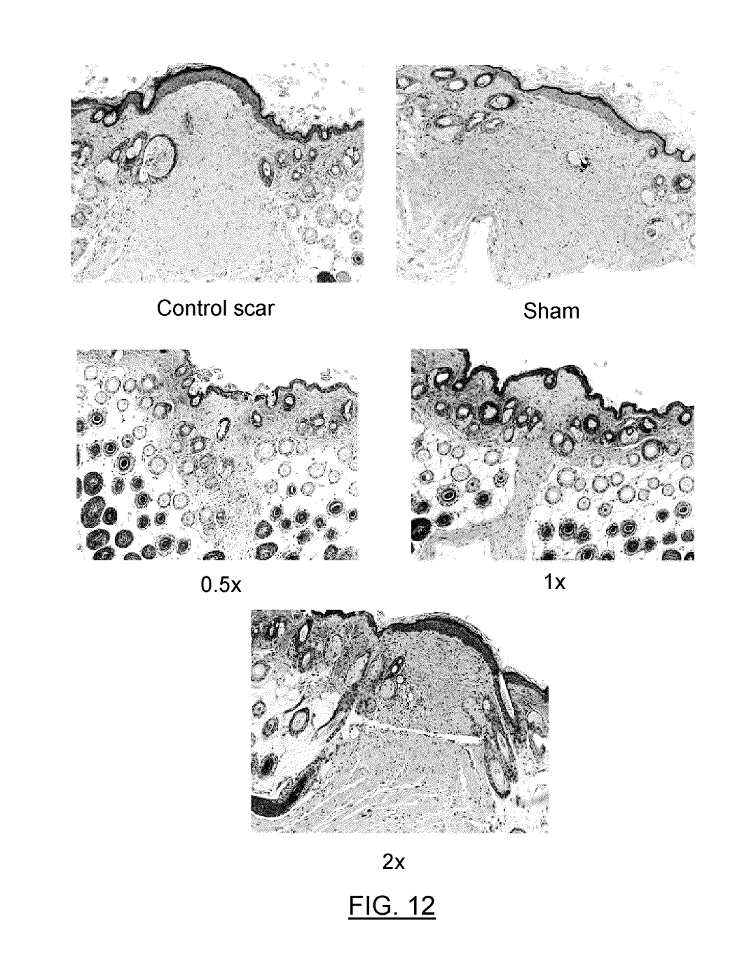

Paraffin embedded sections were stained with Masson's Trichrome in order to better visualize collagen deposition. Sham, control scar and 2× groups showed greater collagen deposition and a thicker dermal scar than the 0.5× and 1× treatment groups FIG. 12. The dermis in unstretched scar had fewer fibroblasts with more collagen between cells when compared with the 0.5× fibroblasts and 2× group where the fibroblasts are closely spaced.

### Cutaneous TGF-B1 Assay

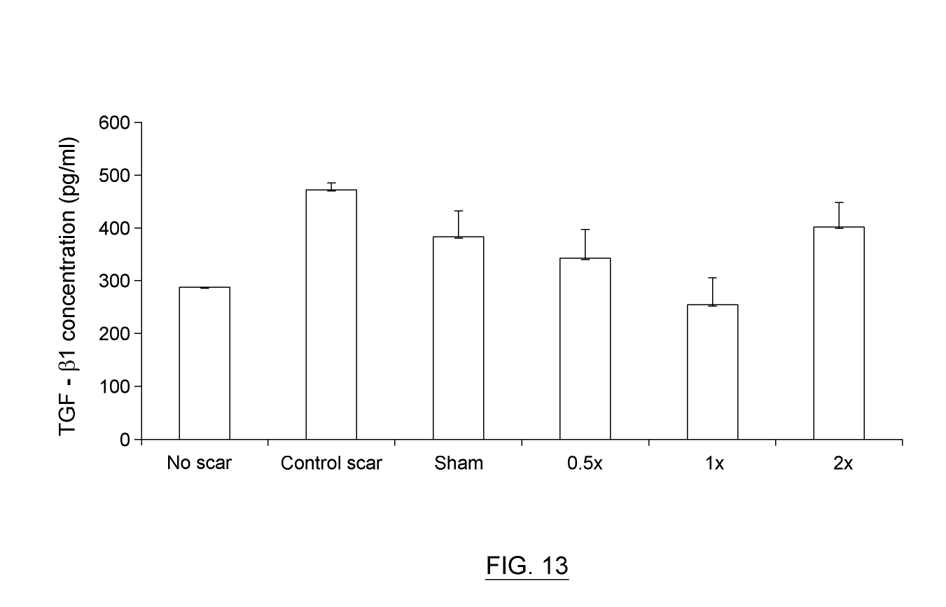

TGF-β1 protein levels in cutaneous scar 20 days after incision were significantly higher in the control scar (471.9±13.8), sham (383.3±49.2) and 2× stretch (401.3±41.1) treatment group. TGF-β1 levels were significantly lower in the stretch treatment groups 0.5× (342.±9 and 1×254.±3, as illustrated in the graph of FIG. 13.

It is worth mentioning that preliminary tests were also conducted on human skin, with satisfactory results. The device was used for a period of 9 days on a closed wound, with a tensile force between 400 g and 800 g.

In summary, the present study confirmed that using a scar reducing device as described above allows reducing scarring of wounds. The study also allowed confirming efficiency of using such device, and of the method for reducing scarring. The study allowed determining the period, timing interval and range of tensile force that need to be applied to a closed wound for obtaining reduced scarring of wound.

Although preferred embodiments of the present invention have been described in detail herein and illustrated in the accompanying drawings, it is to be understood that the invention is not limited to these precise embodiments and that various changes and modifications may be effected therein without departing from the present invention.

1. (canceled)

2. (canceled)

3. The scar reducing device according to claim 17, wherein the first connecting portions comprises one of glue, adhesive, micro-sutures and micro-staples.

4. The scar reducing device according to claim 17, wherein each fastener comprises a one of a hook, loop, magnet button clip, Velcro™ and male/female attachment allowing the first and second connecting portions to be detachably connectable.

5. (canceled)

6. (canceled)

7. (canceled)

8. (canceled)

9. The scar reducing device according to claim 17, wherein the biasing element is a spring having a spring force selected so as to be able to generate the predetermined tensile force in the contracted configuration.

10. The scar reducing device according to claim 17, wherein the biasing element comprises one of an endless screw, a rack and pinion mechanism, a helical spring, opposed pole magnets, an elastomeric element and a resilient metallic or plastic plate, all of which resist axial compression.

11. The scar reducing device according to claim 17, wherein the predetermined tensile force is between 100 g and 2000 g.

12. The scar reducing device according to claim 17, comprising a controlling mechanism for controllably varying the predetermined tensile force.

13. (canceled)

14. The scar reducing device according to claim 17, comprising a sensor for measuring at least one of a biometric characteristic of the skin in the region of the wound and static shear stretch generated by the extension mechanism.

15. The scar reducing device according to claim 14, wherein said biometric characteristic includes temperature, humidity, and tensile strength of the skin.

16. (canceled)

17. A scar reducing device for stretching skin having a closed wound therein so as to reduce scar formation, the scar reducing device comprising:
an extension mechanism including:
first and second elongated members aligned longitudinally with one another, each of said elongated members having inner and outer ends; and
a biasing element disposed between the inner ends of the elongated members, the biasing element being able to generate a predetermined tensile force and allowing displacing the first and second elongated members along a longitudinal axis, thereby moving the extension mechanism from a contracted configuration to an extended configuration,
first and second fasteners removably attachable to skin regions located proximate to the wound, each of said fasteners including a first connecting portion for attachment to the skin, and a second connecting portion affixed to the outer end of a corresponding one of the elongated members, said first and second connecting portions being manually attachable and detachable from one another;
whereby when in use, the fasteners are affixed to said skin regions such that the longitudinal axis is substantially parallel to the wound, the biasing element forcing said first and second fasteners away from one another with the predetermined tensile force, thereby stretching the skin proximate to the wound.

18. The scar reducing device according to claim 17, wherein the extension mechanism includes a tubular body for housing said biasing element, the elongated members telescoping within the tubular body, whereby displacement of the elongated members toward one another allowing compression of the biasing element.

19. The scar reducing device according to claim 17, wherein the extension mechanism comprises an indicator indicative of the tensile force applied to the skin, when in use.

20. Use of the scar reducing device as defined in claim 17 for reducing scarring of a closed wound.

21. The use according to claim 20, wherein the use occurs on skin proximate to a closed wound, for periodic and limited time intervals.

22. A method of reducing scarring of skin having a closed wound, the wound having a segment with a substantially linear direction, the method comprising a step of periodically stretching the skin proximate the wound in a stretching direction which is substantially parallel the linear direction of said segment, with a predetermined tensile force.

23. The method according to claim 22, wherein said substantially linear direction of the wound and said stretching direction form an angle varying between 0 and 45 degrees.

24. The method according to claim 23, wherein said step of stretching the wound is performed during periodic and limited time intervals.

25. The method according to claim 24, wherein the time intervals are between 5 and 20 minutes.

26. The method according to claim 25, wherein said step of periodic stretching begins toward the beginning of the proliferative phase of the wound scarring and ends prior the end of the remodeling period.

27. The method according to claim 26, wherein the predetermined tensile force applied to skin surrounding the wound is between 100 and 2000 g.

28. (canceled)

29. (canceled)

30. (canceled)

31. (canceled)

32. The method according to claim 22, wherein said method is performed over a period varying between 7 and 40 days.

33. (canceled)

34. A method of reducing scarring of skin having an elongated closed wound, comprising the steps of:
a) providing a scar reducing device as defined in claim 17;
b) fastening the first connecting portions to the skin along or at opposite ends of the wound, said connecting portions being spaced apart by a given length interval;
c) placing the extension mechanism in the contracted configuration by displacing the elongated members toward one another, for compressing the biasing element and thereby generating the predetermined tensile force;
d) fastening the second connecting portions to said first connecting portions, respectively;
e) releasing the elongated members, thereby generating a static shear stretch through the wound in the skin;
f) leaving the extension mechanism in place for a predetermined time interval; and
g) detaching the first and second connecting portions of the fasteners, for removing the extension mechanism.

35. The method according to claim 34, wherein in step f), the time interval is between 5 and 20 minutes.

36. The method according to claim 35, wherein steps c) to g) are repeated periodically during the scarring proliferative phase of the wound.

37. The method according to claim 36, wherein steps c) to g) are performed at least once a day.

38. The method according to claim 36, wherein steps c) to g) are performed twice a day.
